(19) United States
(12) Patent Application Publication
(10) Pub. No.: US 2016/0031933 A1
(43) Pub. Date: Feb. 4, 2016

(54) AMINO ACIDS PRODUCED ACCORDING TO A PROCESS OF MECHANOCATALYTIC HYDROLYSIS OF PROTEINS

(71) Applicant: UNIVERSITY OF CENTRAL FLORIDA RESEARCH FOUNDATION, INC., Orlando, FL (US)

(72) Inventor: Richard George BLAIR, Oviedo, FL (US)

(73) Assignee: University of Central Florida Research Foundation, Inc., Orlando, FL (US)

(21) Appl. No.: 14/776,500

(22) PCT Filed: Mar. 14, 2014
(86) PCT No.: PCT/US2014/029677

§ 371 Date: Sep. 14, 2015

Related U.S. Application Data
(60) Provisional application No. 61/784,114, filed on Mar. 14, 2013.

(51) Int. Cl. C07K 1/12

(52) U.S. Cl. CPC C07K 1/122

(57) ABSTRACT

The presently disclosed and/or claimed inventive concept(s) relates generally to processes for the non-aqueous hydrolysis of proteins and/or protein-containing materials and, more particularly but without limitation, to methods for producing amino acids from the non-aqueous solid acid hydrolysis of proteins and/or protein-containing materials. More particularly, but without limitation, the methods disclosed herein for producing amino acids from the solid acid hydrolysis of proteins and/or protein-containing materials are performed in a non-aqueous/solvent-free process. In one particular embodiment, the process of producing such amino acids from proteins and/or protein-containing materials includes, without limitation, the step of mechanocatalytically reacting a solid acid with one or more proteins and/or protein containing materials in a non-aqueous/solvent-free process using the solid acid as a catalyst.

### BACKGROUND

1. Field of the Inventive Concept(s)

The presently disclosed and/or claimed inventive concept(s) relates generally to processes for the non-aqueous hydrolysis of proteins and/or protein-containing materials and, more particularly but without limitation, to methods for producing amino acids from the non-aqueous and solvent-free solid acid hydrolysis of proteins and/or protein-containing materials. More particularly, but without limitation, the methods disclosed herein for producing amino acids from the solid acid hydrolysis of proteins and/or protein-containing materials are performed in a non-aqueous/solvent-free process. In one particular embodiment, the process of producing such amino acids from proteins and/or protein-containing materials includes, without limitation, the step of mechanocatalytically reacting a solid acid with one or more proteins and/or protein containing materials in a non-aqueous/solvent-free process using the solid acid as a catalyst.

2. Background of the Inventive Concept(s)

By-product residues from the production of biofuels and low-value, protein-containing crops represent potentially significant sources of high-value amino acids that can be used in a variety of ways and industries, such as for the production of food for human consumption or as nutritional additives for animal feed. As the world's population continues to grow, the market for amino acids will continue to expand as the need for both humans and animals to receive proper nutrition increases and the world's food supply decreases. As of 2011, the nutraceuticals industry, which sells dietary supplements among other items often containing one or more amino acids, was estimated to have a global market of $151 billion and a projected global market of $207 billion by 2016. As such, an efficient and inexpensive method for producing high-value amino acids is of great interest.

As described, one source of protein-containing material that can be converted into amino acids is the residue(s) produced as a by-product during the production of biofuels, for example ethanol, from crops/biomass that contain protein. Typically, biofuel residues are considered a waste and result in an increase in the overall cost of biofuel production due to the time and expense necessary to dispose of the residues. Thus, profitability of biofuel production could be significantly improved by the development of an efficient and low cost process that can convert the biofuel residue by-products into high-value amino acids that could then be commercially solid for a profit in addition to the biofuels. Additionally, it would also be desirable to efficiently and inexpensively convert a low-value, protein-containing crop, for example soy, into high-value amino acids in order to improve the overall profitability of the crop.

Typically, amino acids are produced from protein-containing materials by either acid hydrolysis or enzymatic hydrolysis. However, neither process is optimal. Generally, the processes for hydrolysis of proteins are characterized by the breaking of the peptide bond between the amino acids to provide free (i.e., individual) amino acids and/or polypeptides comprised of several amino acids still bonded together. While acid hydrolysis can be performed with dilute or concentrated acid, dilute acids require high temperature and pressure, while concentrated acids must be removed from the reaction product and can denature the amino acids during the process. As such, the typically used concentrated and dilute acids require a significant amount of waste water to either dilute the acid and/or wash the acid from the products during the process. Additionally, amino acids and polypeptides produced from enzymatic hydrolysis processes can often have a bitter taste making the resulting amino acids unfavorable for use in food products.

Mechanocatalysis or tribocatalysis is a solid-solid reaction using mechanical force without the addition of solvents, i.e., it is a non-aqueous or solvent-free catalytic reaction. Effective mechanocatalysts are mechanically robust and possess sites that are physically accessible and chemically active. Mechanocatalytic processes also typically do not require external heat. Substantially all of the energy for the reaction comes from the pressures and frictional heating provided by the kinetic energy of milling media moving in a container. In a mechanocatalytic system, it is important that intimate contact between the catalyst and reactant is maintained. Pebble (or rolling) mills, shaker mills, attrition mills, and planetary mills are a few examples of mills that effectively “push” the catalyst into contact with the material to be treated in a mechanocatalytic process. A mechanocatalytic process for converting biomass to soluble sugars is, for example, disclosed in U.S. Ser. No. 11/935,712, U.S. Ser. No. 12/621,741, and U.S. Ser. No. 61/721,316, the entire contents of which are hereby incorporated by reference in their entirety.

As such, disclosed and/or claimed herein are processes and methods for economically, safely, and reliably producing reaction products comprising amino acids from the reaction of a proteinaceous (i.e., protein-containing) material with a solid acid catalyst. More particularly, but without limitation, the processes and methods claimed herein for producing reaction products comprising amino acids are performed in a non-aqueous/solvent-free process. Also disclosed and/or claimed herein are reaction products from such a process that comprise at least one of an amino acid, a polypeptide, or combinations thereof. In one particular embodiment, the process of making such reaction products includes, without limitation, the step of mechanocatalytically reacting a proteinaceous material with a solid acid catalyst.

### SUMMARY OF THE INVENTIVE CONCEPT(S)

The presently disclosed and/or claimed inventive concept(s) encompasses a protein hydrolysate product comprising amino acids and polypeptides produced by a non-aqueous and solvent-free catalytic reaction of a proteinaceous material (i.e., a material of, relating to, consisting of, resembling, or pertaining to protein) and a solid acid catalyst. As disclosed herein, the solid acid material is for example, a clay such as kaolin or bentonite, which has a surface acidity as well as a water content. The resulting products can be useful as additives to food products, nutritional supplements, and for other purposes.

The presently disclosed and/or claimed inventive concept(s) also encompasses a method for the production of a protein hydrolysate comprising amino acids and polypeptides by catalytically reacting at least one proteinaceous material and a solid acid catalyst. The inventors have unexpectedly found that when a solid acid material is combined with a proteinaceous material and agitated in a non-aqueous environment, a high yield of free amino acids can be produced. In the process, the agitation of the material, typically in a mill, provides the kinetic energy necessary to drive the hydrolysis reaction while the solid acid material has a surface acidity that aids in hydrolyzing the peptide bonds of the proteinaceous material. In addition, when the solid acid material has a sufficient existing water content, the water of the solid acid material can provide the water necessary for the hydrolysis reaction without the need for added water or solvent, i.e., the hydrolysis is non-aqueous and solvent-free. For example, in one embodiment of the presently disclosed and/or claimed inventive concept(s), the solid acid material is a clay material, such as kaolin or bentonite, which has a surface acidity as well as a water content. By not requiring traditional aqueous acids, the presently disclosed and/or claimed inventive concept(s) eliminates the large amounts of acid waste and/or degradation products (such as glutamates) that are present in the prior art. The resulting products of the solid acid hydrolysis reaction, which include a quantity of soluble amino acids and polypeptides, are useful as additives to food products and as nutritional supplements, and for other purposes.

In view of the above, in accordance with one aspect of the presently disclosed and/or claimed inventive concept(s), there is provided a method for the production of a protein hydrolysate from at least one proteinaceous material, comprising: (a) contacting the proteinaceous material with a solid acid material; and (b) agitating the proteinaceous material and the solid acid material for a time sufficient to produce a protein hydrolysate reaction product comprising amino acids and/or polypeptides in a solid form. The proteinaceous material may be a pure protein or any other type of protein-containing material and/or source of protein (e.g., any vegetable-based protein or animal-based protein), such as a textured vegetable protein, defatted soy flour, defatted peanut flour, gelatin, and albumin. The solid acid material may be any type of solid or semi-solid material having a surface acidity, defined as H₀, with a value of less than about 1.0, and more preferably less than about −5.6.

Optionally, the above-described method may further comprise: (c) after the step of agitating, recovering the protein hydrolysate comprising amino acids and/or polypeptides in an aqueous solution by rinsing the solid acid material and the proteinaceous material with an aqueous solution. In addition, since the solid acid material is not a reactant in the hydrolysis process, after the step of recovering, the process optionally further comprises: (d) reusing and/or recycling a quantity of the solid acid material back to the reactor and repeating steps (a) and (b), and optionally (c) above, with “fresh” (i.e., additional) proteinaceous material. The process may be performed within a mill or any other suitable vessel that provides agitation of the material therein.

Alternatively, in accordance with another aspect of the presently disclosed and/or claimed inventive concept(s), the method for the production of a protein hydrolysate from at least one proteinaceous material comprises (a) contacting the proteinaceous material with a solid acid material; (b) agitating the proteinaceous material and the solid acid material for a time sufficient to produce a protein hydrolysate reaction product comprising amino acids and/or polypeptides in a solid form, and (c) removing the protein hydrolysate reaction product prior to recycling an amount of the unreacted proteinaceous material back to the reactor wherein additional proteinaceous material is added to the unreacted proteinaceous material and solid acid catalyst to increase the conversion percentage of the original proteinaceous material.

In accordance with another aspect of the presently disclosed and/or claimed inventive concept(s), there is provided a method for the production of a protein hydrolysate comprising soluble amino acids and/or polypeptides from at least one proteinaceous material, comprising:
(a) contacting the proteinaceous material with a solid acid material; and
(b) agitating the proteinaceous material and the solid acid material for a time sufficient to produce a product comprising a protein hydrolysate, wherein agitating occurs at a temperature of between about −5 to about 125 degrees Celsius, and wherein the proteinaceous material and solid acid material have a combined free water content of about 45% or less. The reaction products of those processes contain a protein hydrolysate in a solid form. Thereafter, the method optionally includes steps (c) and (d) as described above.

The presently disclosed and/or claimed inventive concept(s) also contemplates that certain types of solid acid materials may inherently have a water content that enables the hydrolysis of the proteinaceous material to occur without the need for added water. This water may be present as water of crystallization of the solid acid material or materials therein, or as absorbed or adsorbed water of the solid acid material (referred to as the “free water content” below). At least a portion of the water of crystallization may be removed during the steps of agitating as described herein. Moreover, water necessary for the hydrolysis of the protein may be provided by any moisture or water contained in the proteinaceous material. In addition, in the hydrolysis of protein, a dehydration of the peptide bond may take place to provide further water for the hydrolysis reaction. As such, the hydrolysis reaction is disclosed as occurring in a non-aqueous and solvent-free medium, i.e., the water content of the solid acid material and the proteinaceous material combined is less than or equal to 45% by weight.

In accordance with another aspect of the presently disclosed and/or claimed inventive concept(s), during the step (b) of agitating, the free water content of the solid acid material is in the range of about 5% to about 20% by weight of the solid acid material. The free water content of the proteinaceous material and the solid acid material is collectively less than about 45% by weight, and preferably from about 8% to about 40% by weight, so as to not undesirably lower the kinetic energy needed for the hydrolysis reaction upon agitating. By “free water content,” it is meant an amount of water in the proteinaceous material and solid acid containing material that is contained within the proteinaceous material and the solid acid material, but does not pertain to a water of hydration or crystallization of either material. In this way, there is sufficient water in the mixture to drive the hydrolysis reaction. The preferred amount of free water content in the proteinaceous material and the solid acid material is in the range from about 8% to about 22% by weight.

In accordance with yet another aspect of the presently disclosed and/or claimed inventive concept(s), the solid acid material may comprise an aluminosilicate material, such as a clay material. The clay material may be any one of kaolin, bentonite, fuller's earth, or an acid-treated clay material, such as acid-treated bentonite treated with about 1 M hydrochloric acid. When the solid acid material is a clay material, the clay material may have a water content that is attributable to a water of crystallization of the material or materials therein. The water of crystallization may be removed during agitating to further provide needed water for the hydrolysis reaction.

In accordance with still another aspect of the presently disclosed and/or claimed inventive concept(s), the solid acid material may comprise a solid superacid material. Superacids may be defined as acids stronger than 100% sulfuric acid (also known as Brönsted superacids). In addition, superacids may be described as acids that are stronger than anhydrous aluminum trichloride (also known as Lewis superacids). Solid superacids are composed of solid media that are treated with either Brönsted or Lewis acids. In one embodiment, the solid acid is a solid superacid comprising alumina treated with 2 M sulfuric acid, filtered and calcined at about 800° C. for about 5 hours.

In accordance with another aspect of the presently disclosed and/or claimed inventive concept(s), the ratio of the proteinaceous material to the solid acid material is from about 0.5:1 to about 10:1. When the solid acid material is a clay material in one embodiment, the ratio of the proteinaceous material to the solid acid material may be provided in the range of from about 1:1 to about 3:1 because the clay material contains a free water content, as well as water of crystallization.

Proteins are biological molecules consisting of one or more chains of amino acids (i.e., polypeptide chains) that are linked by peptide (i.e., amide) bonds between the carboxyl group of one amino acid and the amino group of another amino acid. Proteins can be described by their primary structure (the amino acid sequence), secondary structure (repeating local structures of peptide chains), tertiary structure (the overall shape of the protein), and the quaternary structure (the structure formed by several protein molecules). Proteins can vary in size depending on the number of amino acids linked together by peptide bonds and also in the sequence of the amino acids. The peptide bonds between the amino acids can be broken by hydrolysis which may result in free (i.e., single) amino acids and/or short peptide chains having two or more amino acids connected by a peptide bond. When traditional acid hydrolysis is used for the production of amino acids from proteinaceous materials, the process produces a reaction product comprising a protein hydrolysate (i.e., free amino acids and polypeptides) and unwanted dehydration by-products (such as glutamates) in addition to an acidic waste. Mechanocatalytic non-aqueous, solvent-free acid hydrolysis performed according to the methods taught herein consistently produces a protein hydrolysate comprising amino acids and polypeptides without the excessive undesirable dehydration by-products and acidic waste produced in the traditional acid hydrolysis method.

In a more specific embodiment, the proteinaceous material reacts with the solid acid catalyst resulting in a conversion of at least 50 percent by weight of the protein in the proteinaceous material into a protein hydrolysate comprising amino acids and/or polypeptides. In an even more specific embodiment, the conversion of the protein in the proteinaceous material to protein hydrolysate comprises at least 60 percent, at least 70 percent, at least 80 percent, at least 90 percent, at least 95 percent, or at least 100 percent. Thus, in view of the teachings herein, those skilled in the art will be able to identify a reaction product produced according to the embodiments of the presently disclosed and/or claimed inventive concept(s). Furthermore, the ability to hydrolyze proteinaceous materials into a protein hydrolysate provides a material comprising a higher yield of free amino acids and/or polypeptides with less dehydration by-products and acidic waster, thereby equating to higher efficiencies and lower cost for producing food additives, nutraceuticals, and other products from amino acids and polypeptides. As the presently disclosed and/or claimed inventive concept(s) are non-aqueous and solvent-free processes, the complexity of the reactions are decreased and the reaction products significantly reduce and/or do not contain dehydration by-products that are detrimental to the commercial products and further downstream processes for obtaining the commercial products.

In an additional specific embodiment, the proteinaceous material reacts with the solid acid catalyst resulting in a conversion of at least 70 percent by weight of the proteinaceous material, which may comprise free water content, fiber, protein, and minerals, into soluble components, wherein at least 50 percent by weight of the proteinaceous material is protein which is converted into a protein hydrolysate comprising free amino acids and polypeptides. In an even more specific embodiment, the proteinaceous material reacts with the solid catalyst resulting in a conversion of at least 80, at least 90, at least 95, or at least 100 percent of the proteinaceous material is converted into soluble components, wherein at least 60, at least 70, at least 80, at least 90, at least 95, or at least 100 percent by weight of the proteinaceous material is protein which is converted into a protein hydrolysate comprising free amino acids and polypeptides.

According to other certain method embodiments, the reaction products possess a reaction profile in regard to the yield of reaction product versus process time. Thus, according to one embodiment, the presently disclosed and/or claimed inventive concept(s) pertains to a reaction product (typically in the form of a powdered composition, solid composition, and/or a liquid suspension) comprising a soluble protein hydrolysate. Accordingly, based on this, and in view of the teachings herein, those skilled in the art will be able to identify a reaction product produced according to embodiments of the presently disclosed and/or claimed inventive concept(s).

### BRIEF DESCRIPTION OF THE DRAWINGS

The inventors have unexpectedly found that when a solid acid material is combined with a proteinaceous material and agitated in a non-aqueous and solvent-free environment, a high yield of free amino acids and polypeptides can be produced.

FIG. 1 is a flow schematic of one embodiment of the non-aqueous and solvent-free solid acid hydrolysis process as disclosed herein.

FIG. 2 is a graphical representation of the percent yield of water soluble reaction products from the hydrolysis of cellulose and textured vegetable protein, and the percent of the protein in the texture vegetable protein converted to protein hydrolysates.

### DETAILED DESCRIPTION OF THE INVENTIVE CONCEPT(S)

Before explaining at least one embodiment of the presently disclosed and/or claimed inventive concept(s) in detail, it is to be understood that the presently disclosed and/or claimed inventive concept(s) is not limited in its application to the details of construction and the arrangement of the components or steps or methodologies set forth in the following description or illustrated in the drawings. The presently disclosed and/or claimed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and/or claimed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed and/or claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed herein can be made and executed without undue experimentation in light of the present disclosure. While the articles and methods of the presently disclosed and/or claimed inventive concept(s) have been described in terms of preferred embodiments, it will be apparent to those of skill in the art that variations may be applied to the articles and/or methods and in the steps or in the sequence of steps of the method described herein without departing from the concept, spirit and scope of the presently disclosed and/or claimed inventive concept(s). All such similar substitutes and modifications apparent to those skilled in the art are deemed to be within the spirit, scope and concept of the presently disclosed and/or claimed inventive concept(s).

As utilized in accordance with the present disclosure, the following terms, unless otherwise indicated, shall be understood to have the following meanings.

The use of the word “a” or “an” when used in conjunction with the term “comprising” may mean “one,” but it is also consistent with the meaning of “one or more,” “at least one,” and “one or more than one.” The use of the term “or” is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

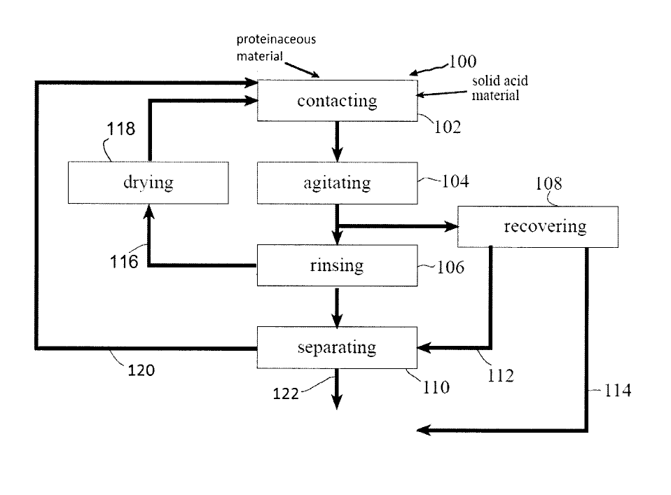

Now referring to the figures, FIG. 1 shows a schematic representation of a process 100 for the production of a protein hydrolysate comprising soluble amino acids and/or polypeptides from a proteinaceous material in accordance with one aspect of the presently disclosed and/or claimed inventive concept(s). The process 100 comprises the hydrolytic conversion of a proteinaceous material to a protein hydrolysate reaction product(s) comprising soluble amino acids and polypeptides. The process 100 is a non-aqueous and solvent-free solid acid hydrolysis reaction. In step 102, a quantity of a proteinaceous material is contacted with a quantity of a solid acid material. To accomplish this, the materials may be introduced into any suitable vessel and, preferably, the vessel in which the step of agitating will take place in step 104, for example, by any suitable method, and simultaneously or sequentially one after the other. While not necessary, it is contemplated that the proteinaceous material may be pretreated as desired, such as by breaking or grinding the material down to a desired size, before bringing the proteinaceous material and solid acid material into contact with one another. In all embodiments, the aggregation of the proteinaceous material and the solid acid material results in a non-aqueous and solvent-free reactant mixture suitable for a non-aqueous and solvent-free acid hydrolysis reaction.

The proteinaceous material may be any material or mixture of materials having a protein content. Thus, in one embodiment, the proteinaceous material may be a purified source of protein, such as albumin, and, may in certain embodiments, comprise greater than 1, 5, 10, 15, 20, 30, 40, 50, 60, 70, 80, 90, 95, or even 100 percent pure protein separated away from any contaminants and/or other reactive and non-reactive materials. In another embodiment, the protein-containing material is a natural protein feedstock, generically referred to herein as a proteinaceous material. Exemplary proteinaceous materials include vegetable-based proteins, animal-based proteins, and more specifically but without limitation, textured vegetable protein, defatted soy flour, defatted peanut flour, gelatin, and albumin. The nature of the proteinaceous material should not be considered to be constraining to the processes and methods disclosed herein. Indeed, the inventors have found to date that all proteinaceous materials that have been tested are suitable and appropriate for the processes and methods disclosed herein.

One embodiment encompasses a method for the production of a protein hydrolysate comprising amino acids and polypeptides by catalytically reacting a proteinaceous material and a solid acid catalyst. The inventors have unexpectedly found that when a solid acid material is combined with a proteinaceous material and agitated in a non-aqueous and solvent-free environment, a high yield of free amino acids can be produced. In the process, the agitation of the material, typically in a mill, provides the kinetic energy necessary to drive the hydrolysis reaction while the solid acid material has a surface acidity that aids in hydrolyzing the peptide bonds of the proteinaceous material. In addition, when the solid acid material has a sufficient existing water content, the water of the solid acid material can provide the water necessary for the hydrolysis reaction without the need for added water or solvent, i.e., the hydrolysis is non-aqueous and solvent-free. For example, in one embodiment of the presently disclosed and/or claimed inventive concept(s), the solid acid material is a clay material, such as kaolin or bentonite, which has a surface acidity as well as a water content. By not requiring traditional aqueous acids, the presently disclosed and/or claimed inventive concept(s) eliminates the large amounts of acid waste and/or degradation products (such as glutamates) that are present in the prior art. The resulting products of the solid acid hydrolysis reaction, which include a quantity of soluble amino acids and polypeptides, are useful as additives to food products and as nutritional supplements, and for other purposes. Any quantity of proteinaceous material may be provided and used in the presently disclosed and/or claimed inventive concept(s) and the particular ratios of reactants disclosed herein should be considered as not-limiting examples and/or specific embodiments.

The solid acid material may be any solid material having a surface acidity. By “solid,” it is meant a solid material, a semi-solid material, or any other material having a water content of less than about 45% by weight, or more preferably less than about 40% by weight. Surface acidity refers to the acidity of the solid surface of the material. Surface acidity determination methods are founded on the adsorption of a base from the base's solution. The amount of base that will cover the solid surface of the solid acid material with a monolayer is defined as the surface acidity and corresponds to the pKₐ of the base used. The base used may be n-butylamine, cyclohexamine, or any other suitable base. The degree of surface acidity is typically expressed by the Hammet and Deyrups H₀ function.

H₀=pK_{BH+}−log(C_{BH+}/C_{B}) (I)

Thus, according to equation I, when an indicator, B, is adsorbed on an acid site of the solid surface of the material, a part of the indicator is protonated on the acid site. The strength of the acid sites may be represented by equation (I) by the value of pK_{BH+} of BH⁺. BH⁺ is the conjugate acid of indicator B when the concentration of BH⁺ (C_{BH+}) is equal to the concentration of B (C_{B}). Therefore, the acid strength indicated by H₀ shows the ability of the conjugate to change into the conjugate acid by the acid sites that protonates half of the base indicator B. Under a Lewis definition, the H₀ value shows the ability that the electron pair can be received from half of the absorbed base indicator B. See, Masuda et al., Powder Technology Handbook, 3^{rd} Ed. (2006). A H₀ of −8.2 corresponds to an acidity of 90% sulfuric acid and a H₀ of −3.0 corresponds to an acidity of about 48% sulfuric acid.

Any suitable method of determining the H₀ of the solid acid material may be used, such as the method using the adsorption of n-butylamine from its solution in cyclohexane as set forth in Investigation of the Surface Acidity of a Bentonite modified by Acid Activation and Thermal Treatment, Turk. J. Chem., 2003; 27:675-681. Alternatively, indicators, generally referred to as Hammett indicators, may be used to determine the H₀ of a material. Hammett indicators rely on color changes that represent a particular surface acidity of the subject material. In the presently disclosed and/or claimed inventive concept(s), any solid acid material having a surface acidity can be used although a number of solid acid materials have been found to be particularly beneficial. For example, it has been found that a solid acid material having an H₀ of less than about −3.0, and preferably less than about −5.6 is particularly useful in the processes and methods disclosed herein.

In one embodiment, the solid acid material may comprise a clay material. As used herein, “a clay material” is defined as a material composed primarily of fine-grained minerals, which is generally plastic at appropriate water contents and will harden when dried or fired in, for example, a kiln. Exemplary minerals that comprise the major proportion of clay materials for use in the presently disclosed and/or claimed inventive concept(s) include kaolinite, halloysite, attapulgite, montmoirllonite, illite, nacrite, dickite, and anauxite. Non-limiting examples of clays for use in the presently disclosed and/or claimed inventive concept(s) include fuller's earth, kaolin, bentonite, and combinations thereof. Kaolin is a clay material that mainly consists of the mineral kaolinite. Bentonite is a clay containing appreciable amounts of montmorillonite, and typically having some magnesium associated therewith. Fuller's earth usually has a high magnesium oxide content in combination with montmorillonite or palygorskite (attapulgite) or a mixture of the two; additional minerals that may be present in fuller's earth deposits are calcite, dolomite, and quartz. Optionally, the clay material may be acid-treated to provide further surface acidity to the clay material in addition to its inherent acidic properties. Alternatively, the clay material may be treated with a base or other agent to lower the surface acidity of the clay material. It should be understood that the surface acidity can be tailored to meet specific needs or embodiments of the presently disclosed and/or inventive concepts and such tailoring is well within the abilities of a skilled artisan.

In another embodiment, the solid acid material may comprise any aluminosilicate or hydrated aluminosilicate mineral. For example, the solid acid may comprise vermiculite, muscovite mica, kaolinite, halloysite, attapulgite, montmorillonite, illite, nacrite, dickite, and anauxite, or zeolites such as analcime, chabazite, heulandite, natrolite, phillipsite, and stilbite, or any mineral having the general formula Al₂O₃.xSiO₂.nH₂O, and combinations thereof.

In another embodiment, the solid acid material may comprise a superacid material. Superacid materials are useful in the presently disclosed and/or claimed inventive concept(s) because of the high number of acidic sites on the surface of the superacid material. Brönsted superacids may be described as acids which are stronger than 100% sulfuric acid. Lewis superacids may be described as acids that are stronger than anhydrous aluminum trichloride. Solid superacids are composed of solid media, e.g., alumina, treated with either Brönsted or Lewis acids. The solids used may comprise natural clays and minerals, metal oxides and sulfides, metal salts, mixed metal oxides, and combinations thereof. Exemplary Brönsted superacids include titanium dioxide:sulfuric acid (TiO₂:H₂SO₄) and zirconium dioxide:sulfuric acid (ZrO₂:H₂SO₄) mixtures. Exemplary Lewis superacids involve the incorporation of antimony pentafluoride into metal oxides, such as silicon dioxide (SbF₅:SiO₂), aluminum oxide (SbF₅:Al₂O₃), or titanium dioxide (SbF₅:TiO₂). In one embodiment, the superacid comprises a metal oxide treated with either Brönsted or Lewis acids. In a particular embodiment, the superacid comprises alumina treated with sulfuric acid as set forth below. Alternatively, the solid acid material may comprise a silicate material, such as talc or any other suitable solid material having a surface acidity, such as alumina, and combinations of any of the materials described herein.

Kaolin is composed primarily of the mineral kaolinite (Al₂Si₂O₅(OH)₄) which is a layered silicate made of alternating sheets of octahedrally coordinated aluminum and tetrahedrally coordinated silicon that are bonded by hydroxyl groups. Alternatively, the solid acid material may be in the form of anhydrous kaolin, which may be prepared by heating kaolin at about 800° C. for at least about 6 hours and preferably at about 800° C. for about 8 hours.

In another embodiment, the solid acid material may comprise bentonite, and preferably acidified bentonite. Bentonite is an absorbent aluminum phyllosilicate clay material consisting mostly of montmorillonite, (Na,Ca)_{0.33}(Al,Mg)₂Si₄O₁₀(OH)₂.(H2O)ₙ. Two types of bentonite exist: swelling bentonite which is also called sodium bentonite and non-swelling bentonite or calcium bentonite. Preferably for use with the presently disclosed and/or claimed inventive concept(s), the solid acid material comprising bentonite is non-swelling bentonite. If an acidified bentonite is chosen as the solid acid material, it may be prepared, for example, but not by way of limitation, by treating bentonite with one or more acids. Particularly, bentonite may be treated with a 1 M hydrochloric acid solution thereby providing an acidified bentonite material for use as the solid acid material. In still another particular embodiment, the solid acid material may be a solid superacid comprising alumina treated with 2 M sulfuric acid, filtered and calcined at about 800° C. for about 5 hours.

Without wishing to be bound by any particular method of reaction, it is believed that the kaolin and acidified bentonite are particularly useful as the solid acid material for use in the presently disclosed and/or claimed inventive concept(s) because they provide a high surface acidity along with an inherent amount of water with the material. Such water is a function of the materials' inherent water of crystallization and a free water content. Such an inherent water content is useful and necessary to hydrolyze the peptide bonds of the proteinaceous material in the solid acid hydrolysis process presently disclosed and/or claimed herein. Although the solid acid material has an inherent water content, it should be understood that the reactants, either alone or in combination, are still to be considered in a solid or non-aqueous phase. Therefore, whether using acidified bentonite, bentonite, and/or kaolin as the solid acid material, a non-aqueous and solvent-free solid acid hydrolysis of a proteinaceous material can be performed, i.e., a hydrolysis reaction can undergo even in a non-aqueous and/or solvent-free environment thereby providing a substantial benefit. Additional water is not required for the hydrolysis reaction to be completed and the time and expense of hydrolyzing the proteinaceous material and extracting and/or separating the reaction products from unreacted materials downstream of the reactor.

Kaolin and bentonite generally have a free water content of greater than about 4% by weight, as well as a water of crystallization content. Accordingly, in one embodiment, the free water content of the solid acid material is from about 4% to about 10% by weight. Water of crystallization refers to water that occurs as a constituent of crystalline substances in a definite stoichiometric ratio. This water can be removed from the substances by the application of heat at about 700° C., for example, but not by way of limitation, and its loss usually results in a change in the crystalline structure. In the presently disclosed and/or claimed inventive concept(s), it is believed that the agitating step 104 (as described herein) provides the localized heat necessary to remove the water, including the water of crystallization, from the solid acid material (when water of crystallization is present) and thereby provide any water required for the hydrolysis of the proteinaceous material. As such, the solid acid material is, in effect, providing both the catalytic acid functionality as well as at least a portion of the water required for the hydrolysis reaction of the proteinaceous material to take place.

The water content of most compounds, including the water of crystallization of the solid acid material, can be determined by thermogravimetric analysis (TGA), where the sample is heated, and the accurate weight of a sample is plotted against the temperature. Alternatively, any other suitable method for determining water content of the solid acid material may also be used, including mass loss on heating, Karl Fischer filtration, and freeze drying, or any other suitable method. As such, one of ordinary skill in the art would be readily capable of determining if a particular solid acid material had a water content of from about 4% to about 10% as disclosed in one particular embodiment of the presently disclosed and/or claimed inventive concept(s). It is believed that kaolin has a similar free water content relative to bentonite and would exhibit similar mass loss corresponding to the adsorbed water and/or the water of crystallization being removed. As discussed above, the inherent water of crystallization and free water content of the solid acid material and, in particular embodiments, the clay materials disclosed herein, is useful for the hydrolysis of the peptide bond in the processes of the presently disclosed and/or claimed inventive concept(s).

In another embodiment, the solid acid material may comprise an acid-treated material, such as sulfuric acid-treated alumina, to form a superacid. To prepare this superacid, alumina was stirred in 2 M sulfuric acid, filtered and calcined at about 800° C. for about 5 hours. Treating the alumina with sulfuric acid adds sulfate ions to the solid alumina surface, thereby allowing the solid acid material to further accept electrons. As a result, these superacids have a very high surface acidity. However, while superacids may have a higher surface acidity than bentonite or kaolinite, the superacids may not have as much inherent water as the bentonite and/or kaolinite solid acid materials. As a result, while not wishing to be bound by theory, it appears that the additional water content found in kaolin and bentonite contributes to a higher solubilization efficiency for protein within a proteinaceous material when reacted with a solid acid material in a non-aqueous environment. This statement is further supported in showing that the solubilization efficiency is lower for anhydrous kaolinite which has a lower inherent water content than kaolin.

The ratio of the proteinaceous material to the solid acid material is such that the solubilization of the proteinaceous material is optimized. Generally, the solubilization efficiency is optimized by determining a ratio of the proteinaceous material to the solid acid material, wherein a surface interaction of the solid acid material and the proteinaceous material is maximized and the combined inherent water content of the proteinaceous material and solid acid material is optimized. If there is too much moisture in the combined proteinaceous material and the solid acid material, or in the individual materials themselves, during the agitating step 104, the amount of kinetic energy available to drive the hydrolysis of protein is lowered and the overall process results in a lowered yield of reaction products, i.e., solid and/or powdered soluble amino acids and polypeptides. On the other hand, incomplete solubilization of the proteinaceous material and protein therein results if the water content is too low. As such, it should be appreciated to a skilled artisan that there must exist at least some inherent water content in the proteinaceous material and the solid acid material, alone or in combination, in order for the hydrolysis reaction to occur. It should be understood, however, that the existence of such an amount of inherent water in the reactants should not be interpreted to mean that the reaction (i.e., the agitating step 104) occurs in an aqueous environment: rather, while requiring some minor amount of water, the hydrolysis reaction is being carried out in a non-aqueous and solvent-free environment and the proteinaceous material and the solid acid material should be considered to be in a solid form.

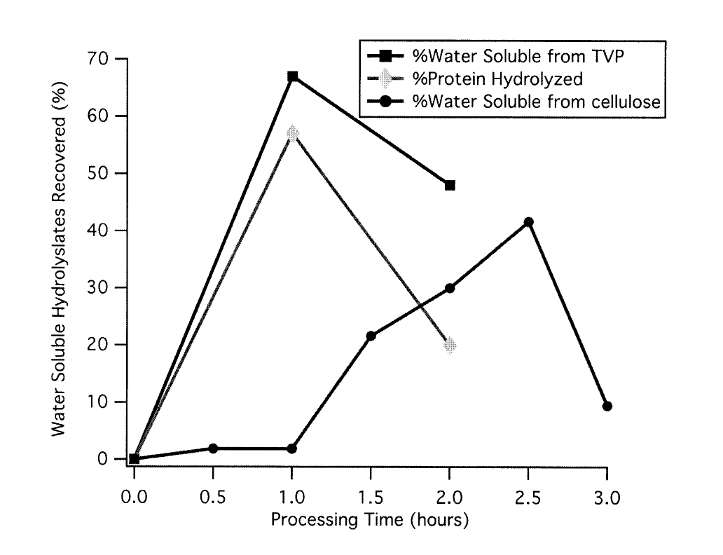

In one embodiment, the proteinaceous material is provided in a ratio of from about 0.5:1 to about 10:1 proteinaceous material to solid acid material. In a particular embodiment, when the solid acid material is kaolin for example, FIG. 2 indicates that at least one optimal yield of reaction product containing solid and/or powdered soluble protein hydrolysate comprising amino acids and polypeptides is obtained with about a 1:1 mass ratio of textured vegetable protein (Bob's Red Mill, Milwaukie, Oreg.) to kaolin (Edgar Minerals, Putnam, County, Fla.) after about 1 hour of milling in a UNION PROCESS® 01-HDDM attrition mill (Union Process, Inc., Akron, Ohio) wherein the attritor agitator (‘tree’) was rotated at 600 rpm. The reactants were nominally dry with a combined free water content of at least 10 percent by weight of the reactants. The reactants were milled in one hour increments for a total of three hours in 1.4 L milling tanks constructed of 304 stainless steel with 6 lbs. of 440C steel balls ¼″ in diameter being used as a milling media. The reactants and the milling media are being agitated in step 104 in a non-aqueous environment and both the reactants and the reaction product should be considered as being in a solid form. As shown in FIG. 2, the percent yield of the protein hydrolysate beings to decrease after 1 hour, the percent yield of the overall water soluble components from the textured vegetable protein decreases after two hours, and the percent yield of the water soluble components from the cellulose decreases after 3 hours. This is due to water loss and the formation of insoluble dehydration products. This information provides the feedback necessary to control the solid acid catalyzed hydrolysis of the proteinaceous material such as to avoid producing unwanted insoluble dehydration products, thereby avoiding costly waste and increasing the efficiency of the process of hydrolyzing proteinaceous materials to obtain the valuable amino acids.

In one embodiment, the proteinaceous material may have a free water content of from about 4% to about 40% by weight of the proteinaceous material. Thus, when the proteinaceous material and the solid acid material are contacted in step 102 and agitated in step 104, in one specific non-limiting embodiment, the free water content of the collective mixture of the reactants (i.e., the inherent water of the solid acid material and the protein and/or proteinaceous material) should be less than about 45% by weight of the materials (thereby maintaining the reactants in a solid and/or non-aqueous environment), and, more preferably, the free water content of the collective mixture of the reactants is less than about 30% by weight, less than about 20% by weight, less than about 10% by weight, and from about 4 to about 8% by weight. In all embodiments, the free water content of the collective mixture of the reactants is from about 4% to about 40% and, more particularly, from about 8% to about 40% by weight. As described, a sufficient water content is provided by the solid reactants in the non-aqueous and solvent-free environment to hydrolyze the protein (separately or as part of the proteinaceous material) to a reaction product containing solid and/or powdered soluble amino acids and/or polypeptides. It is also contemplated that the process 100 may be performed at ambient temperature (although, the term “ambient” should be understood as the purposeful absence of heating or cooling,—it is contemplated that the reactants and reaction mixture may autogenously provide additional heat through exothermic reactions). Additionally, it is contemplated that the process 100 be performed without the addition of water to the reactant mixture. Of course, although the process is disclosed and described as occurring in a non-aqueous and solvent-free environment, the water content of the reactant mixture may be up to about 45% by weight, or more preferably about 40% by weight and yet still be considered as comprising a non-aqueous mixture. As such, it may be desirable in some situations to add some amount of water to the reactant mixture, e.g., if the reactants have a combined free water content less than or about 4% or if any particular reactant mixture of proteinaceous material and solid acid material requires a greater amount of water than is inherently in the mixture due to the reactants themselves. It should be considered, however, that a significant advantage of the process 100 is that no additional water is generally required for the solid acid material to hydrolytically catalyze the proteinaceous material into a reaction product containing solid and/or powdered soluble amino acids and/or polypeptides. As would be readily apparent to one of ordinary skill, the ability to perform the process 100 according to the presently disclosed and/or claimed inventive concept(s) provides an efficient and effective means of producing a reaction product containing solid and/or powdered soluble amino acids and/or polypeptides on a large commercial batch or continuous manufacturing scale.

In step 104, the proteinaceous material and the solid acid material are agitated for a time sufficient to provide a reaction product containing solid and/or powdered soluble amino acids and/or polypeptides. The agitation may take place in any suitable vessel or reactor. In one embodiment, the agitating step 104 takes place in a ball, roller, jar, hammer, or shaker mill. The mills generally grind samples by placing them in a housing along with one or more grinding elements and imparting motion to the housing. The housing is typically cylindrical in shape and the grinding elements and/or milling media (as discussed above) are typically steel balls, but may also be rods, cylinders, or other shapes. Generally, the containers and grinding elements are made from the same material.

As the container is rolled, swung, vibrated, or shaken, the inertia of the grinding elements and/or milling media causes the milling media to move independently into each other and against the container wall, grinding the proteinaceous material and the solid acid material and bringing the reactants into reactive contact with one another. In one embodiment, the mill is a shaker mill using steel balls as the milling media and shaking to agitate the proteinaceous material and the solid acid material. The mills for use in the presently disclosed and/or claimed inventive concept(s) may range from those having a sample capacity of a gram or less to large industrial mills with a throughput of tons per minute. Such mills are available from SPEX CertiPrep of Metuchen, N.J., for example, Paul O. Abbe, Bensenville, Ill., or Union Process Inc., Akron, Ohio. For some mills, such as a steel ball mill from Paul O. Abbe, the optimal fill volume is about 25% of the total volume of the mill. The number of steel balls (i.e., the milling media) required for the process 100 is typically dependent upon the amount of kinetic energy available. High energy milling like that in a shaker mill will require less milling media than lower energy milling methods such as rolling mills. For shaking mills, a ball to sample mass ratio (i.e., a milling media to reactant mass ratio) of about 12:1 is sufficient. For rolling mills, a ball to sample mass ratio (i.e., a milling media to reactant mass ratio) of about 50:1 works well for a rolling rate of about 100 rpm. Lower mass ratios can be obtained by increasing the amount of kinetic energy available to the system. In a roller mill, this can be achieved through the optimization of mill geometry and/or increasing the mill's rotational velocity.

A significant advantage of the presently disclosed and/or claimed inventive concept(s) is that the processes described herein can be performed at ambient temperature without the need for added heat, cooling, or modifying pressure. Instead, the processes, including the agitation step, can be performed under ambient conditions. Without wishing to be bound by theory, it is believed the agitating step 104 of the proteinaceous material with the solid acid material, such as in with the aforementioned mills, provides the process with the energy required for the hydrolysis of the protein in the proteinaceous material. Moreover, it is believed the agitating step 104 also allows more of the proteinaceous material to contact the acidic sites on the surface of the solid acid material. Even further, it is believed that the heat created by the agitating step 104 frees the inherent water content of the reactants to provide the water necessary for the hydrolysis reaction to take place. In an alternate embodiment, the agitating step 104 may occur at a controlled temperature of between about −5 to about 125 degrees C., or more preferably from about −5 to about 105 degrees C. It is contemplated that the agitating step 104 may occur at any temperature degree value within this range (rounded to the nearest 0.5 centigrade unit), or within any sub-ranges within this range (rounded to the nearest 0.5 centigrade unit).

After the step of agitating 104, the reaction products may be optionally washed with a first aqueous solution and resulting solubilized reaction products can be recovered in step 108. Typically, this aqueous solution will comprise an aqueous solution of a protein hydrolysate reaction product containing solid and/or powdered soluble amino acids and/or polypeptides. It is contemplated that the first aqueous solution may also comprise other byproducts of the decomposition reactions which occur during the agitating step 104, such as glutamates.

Preferably, after the step of agitating 104, the proteinaceous material and solid acid material may be separately rinsed with a second aqueous solution as set forth below in step 106. Alternatively, from recovering step 108, at least a portion of the first aqueous solution is optionally directed to a separating step 110 as indicated by arrow 112, where any separation of the components of the first aqueous solution can be performed by any suitable technique known in the art. Further alternatively, at least a portion of the first aqueous solution may be directed to a further process step as indicated by arrow 114. Additionally, one embodiment encompasses recovering the protein hydrolysate after the agitating step 104 without rinsing the protein hydrolysate with any aqueous solution and therefore quickly move through the recovering step 108 and either separate the reaction products and/or the unreacted reactants and recyclable solid acid catalyst in step 110 or direct the entire mixture to a further processing step as indicated by arrow 114. Arrow 122 indicates the separate reactants and/or reaction products that leave the separating step 110. In a further embodiment, the separated unreacted reactant, e.g., the proteinaceous material not hydrolyzed and/or the recyclable solid acid catalyst can be recycled back into the contacting container 102 as indicated by arrow 120, where it is then optionally mixed with new (i.e., “fresh”) proteinaceous material. The solid acid catalyst can be recycled at least 8 times through the process.

When using a mill as described herein, the hydrolysis processes described herein are generally carried out as a batch process. In addition, the vessel where the agitating and hydrolysis reaction takes place may be performed in a continuous attritter, which is commercially available from Union Process, Akron, Ohio. This device more generally allows the process to be carried out as a continuous process.

The milling time performed in the agitating step 104 may have an effect on the extent of solubilization of the proteinaceous material. For example, as shown in FIG. 1, the textured vegetable protein reaches a maximum percent of solubilization after about one hour in an attrition mill using 6 lbs. of ¼″ diameter 440C steel balls as milling media.

It is contemplated that at least about 55% of the available protein in the proteinaceous material may be hydrolyzed to a protein hydrolysate reaction product containing solid and/or powdered soluble amino acids and/or polypeptides after a single pass of the proteinaceous material through the process in various embodiments of the present invention. Additionally, it is contemplated that at least 96% of the available protein in an amount of proteinaceous material may be hydrolyzed to a protein hydrolysate reaction product containing solid and/or powdered soluble amino acids and/or polypeptides after multiple passes of the unreacted available protein of the proteinaceous material through the process with new (i.e., “fresh”) proteinaceous material added to the unreacted proteinaceous each pass. In one specific embodiment, 4 passes of the proteinaceous material through the process resulted in a conversion of at least 96% by weight of the proteinaceous material into a protein hydrolysate comprising amino acids and polypeptides. It is appreciated that higher efficiencies may be obtained by selecting the various solid acids, milling time, and modifying the ratio of the proteinaceous material to the solid acid material. If relatively pure protein is used, it is contemplated that less proteinaceous material may be required than if the proteinaceous material were a low protein-containing material.

Referring again to FIG. 1, after step 104 of agitating, the proteinaceous material and solid acid material may be washed with a second aqueous solution in step 106 to produce a second aqueous solution comprising reaction product containing solid and/or powdered soluble amino acids and/or polypeptides. Any suitable method of determining the amount of solubilized amino acids and/or polypeptides may be used, such as by chromatographic methods well known in the art. Moreover, the presence of particular solubilized amino acids and/or polypeptides may be confirmed by any suitable chromatography method, such as thin-layer chromatograph, gas chromatography (GC), high-performance liquid chromatography (HPLC), GC-MS, LC-MS, or any other suitable method known in the art. The second aqueous solution may also comprise by-products, such as glutamates, as previously described.

The washing step 106 may be repeated until it is relatively certain that the bulk of the reaction product containing solid and/or powdered soluble amino acids and/or polypeptides have been recovered in the second aqueous solution. Thereafter, the second aqueous solution may be directed to the separating step 110 for separation of any of the desired components by any suitable technique known in the art or, alternatively, the second aqueous solution can bypass the separating step 110 and head to a further processing step without separating the components in the second aqueous solution.

As described above, since the solid acid material is acting as a catalyst in the hydrolysis of the proteinaceous material, the solid acid material may be recycled. Thus, optionally, the solid acid material may be directed to drying step 118 to dry the material to a suitable moisture content, if necessary, as shown by arrow 116, and a new quantity of proteinaceous material can be combined with all or a portion of the recycled solid acid material (and/or portion of the unreacted proteinaceous material recycled after the separation step 110 as shown by the arrow 120) to again produce a quantity of soluble amino acids and/or polypeptides. If no drying step is necessary, the rinsed solid acid material can be immediately reused in contacting step 102. In either instance, the rinsed solid acid material is optionally recycled and reused to hydrolyze further proteinaceous material by starting the process again at step 102. Additional solid acid material may be added as needed to supplement the recycled solid acid material when redoing step 102. Accordingly, a significant advantage of the presently disclosed and/or claimed inventive concept(s) is that at least a portion of the solid acid material may be reused continuously, thereby saving considerable material and expense.

The recovered protein hydrolysate comprising amino acids and/or polypeptides from step 108, any portion of the first and/or second aqueous solutions, or all of the first and/or second aqueous solutions having the soluble, may then be sent to further processing steps as indicated by arrows 122 and 114.

### EXAMPLES

As graphically represented by FIG. 2, a solid acid catalyzed hydrolysis of a proteinaceous material and a solid acid catalyst (collectively, “reactants”) was performed in, and agitation was supplied by, a UNION PROCESS® 01-HDDM attrition mill (Union Process, Inc., Akron, Ohio). The reactants were combined as 1:1 mass ratio of textured vegetable protein (Bob's Red Mill, Milwaukie, Oreg.) to kaolin (Edgar Minerals, Putnam County, Fla.), more particularly, 200 g of the textured vegetable protein was combined with 200 g of the kaolin based solid acid catalyst in the attrition mill, and were thereafter subjected to about 1 hour of milling in the UNION PROCESS® 01-HDDM attrition mill (Union Process, Inc., Akron, Ohio) wherein the attritor agitator (‘tree’) was rotated at 600 rpm. The reactants were nominally dry prior to combining them in the attrition mill with a combined free water content of at least 10 percent by weight of the reactants. The reactants were milled in one hour increments for a total of three hours in 1.4 L milling tanks constructed of 304 stainless steel with 6 lbs. of 440C steel balls ¼″ in diameter being used as a milling media. Additionally, a 1:1 mass ratio of microcrystalline cellulose (Alfa Aesar, Ward Hill, Mass.) to kaolin (Edgar Minerals, Putnam County, Fla.), was also subjected to solid acid catalyzed hydrolysis in the method described above to produce a reaction product comprising soluble sugars.

As can be appreciated from FIG. 2, the percent yield of the protein hydrolysate begins to decrease after 1 hour, the percent yield of the overall water soluble components from the textured vegetable protein decreases after 2 hours, and the percent yield of the water soluble components from the cellulose decreases after 3 hours. This is due to water loss and the formation of insoluble dehydration products. This information provides the feedback necessary to control the solid acid catalyzed hydrolysis of the proteinaceous material such as to avoid producing unwanted insoluble dehydration products, thereby avoiding costly waste and increasing the efficiency of the process of hydrolyzing proteinaceous materials to obtain the valuable amino acids. FIG. 2 also suggests that upon a single pass of proteinaceous material through the process subject to the conditions outlined above, approximately 57% of the available protein in the proteinaceous material is converted to the desired reaction products, amino acids and/or polypeptides, after only an hour.

### 1. Gravimetric Analysis

The extent of hydrolysis of the proteinaceous material and cellulose-containing material was monitored gravimetrically. Conversion of the proteinaceous material and cellulose-containing material to their respective reaction products was determined by stirring 0.1 g of each reaction mixture in 30 mL of water. Any oligosaccharide with a degree of polymerization<5 will be solvated. Additionally, peptides with less than 5 residues (i.e., peptides having less than 5 amino acids) will also be solvated. The production of water-soluble products was measured by filtration through a 47 mm diameter Whatman Nuclepore® track etched polycarbonate membrane filter with a pore size of 0.220 μm. The residue was dried in a 60° C. oven for 12 hours and then weighed.

### 2. Gas Chromatography With Mass Sensitive Detection

GC-MS analysis was performed on an Agilent 6850 GC with an Agilent 19091-433E HP-5MS column (5% phenyl methyl siloxane, 30 m×250 μm×0.25 μm nom.) coupled with a 5975C VL mass selective detector. Saccharide composition and protein hydrolysate composition were analyzed by silanizing each reaction product. Dehydration products were extracted with 60.0 chloroform and analyzed by GC-MS.

The presently disclosed and/or claimed inventive concept(s), in various embodiments, includes components, methods, processes, systems and/or apparatus substantially as depicted and described herein, including various embodiments, subcombinations, and subsets thereof. Those of skill in the art will understand how to make and use the presently disclosed and/or claimed inventive concept(s) after understanding the present disclosure. The presently disclosed and/or claimed inventive concept(s), in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

The foregoing discussion of the presently disclosed and/or claimed inventive concept(s) has been presented for purposes of illustration and description. The foregoing is not intended to limit the presently disclosed and/or claimed inventive concept(s) to the form or forms disclosed herein. In the foregoing Detailed Description for example, various features of the presently disclosed and/or claimed inventive concept(s) are grouped together in one or more embodiments for the purpose of streamlining the disclosure. This method of disclosure is not to be interpreted as reflecting an intention that the claimed presently disclosed and/or claimed inventive concept(s) requires more features than are expressly recited in each claim. Rather, as the following claims reflect, presently disclosed and/or claimed inventive concept(s) lie in less than all features of a single foregoing disclosed embodiment. Thus, the following claims are hereby incorporated into this Detailed Description, with each claim standing on its own as a separate preferred embodiment of the presently disclosed and/or claimed inventive concept(s).

Moreover, though the description of the presently disclosed and/or claimed inventive concept(s) has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A protein hydrolysate reaction product produced by a non-aqueous and solvent-free hydrolysis reaction of at least one proteinaceous material and a solid acid material.

2. The protein hydrolysate reaction product of claim 1, wherein the protein hydrolysate reaction product is produced by a mechanocatalytic reaction of the at least one proteinaceous material and the solid acid material.

3.-5. (canceled)

6. The protein hydrolysate reaction product of claim 1, wherein the solid acid material is selected from the group consisting of aluminosilicate materials, solid superacid materials, and combinations thereof.

7. The protein hydrolysate reaction product of claim 1, wherein the solid acid material comprises an acid treated clay material, wherein the clay material is selected from the group consisting of kaolin, bentonite, fuller's earth, and combinations thereof.

8.-9. (canceled)

10. The protein hydrolysate reaction product of claim 6, wherein the solid acid material comprises a solid superacid material, wherein the solid superacid material comprises a solid selected from the group consisting of natural clays, natural minerals, metal oxides, metal sulfides, metal salts, and combinations thereof that has been treated with at least one of a Bronsted acid and a Lewis acid.

11.-12. (canceled)

13. The protein hydrolysate reaction product of claim 1, wherein the solid acid material has a surface acidity, as expressed by the Hammet and Dryups function value, Hₒ, of less than about 1.

14. (canceled)

15. The protein hydrolysate reaction product of claim 1, wherein the at least one proteinaceous material and the solid acid material have a combined free water content less than about 45% by weight.

16.-17. (canceled)

18. The protein hydrolysate reaction product of claim 1, wherein the protein hydrolysate reaction product comprises at least one of an amino acid, a polypeptide, and combinations thereof, and wherein the protein hydrolysate reaction product is substantially free of glutamates.

19. (canceled)

20. The protein hydrolysate reaction product of claim 1, wherein at least about 50% by weight of the protein in the proteinaceous material is converted to a protein hydrolysate comprising at least one of an amino acid, a polypeptide, and combinations thereof.

21. The protein hydrolysate reaction product of claim 1, wherein at least 70% by weight of the proteinaceous material is converted to soluble components, and further wherein at least 50% by weight of the proteinaceous material is protein converted to a protein hydrolysate comprising at least one of an amino acid, a polypeptide, and combinations thereof.

22. A method for the production of a protein hydrolysate reaction product, comprising the step of hydrolytically reacting a solid acid material and at least one proteinaceous material in a non-aqueous and solvent-free environment for a period of time sufficient to produce the reaction product.

23. (canceled)

24. The method of claim 22, wherein the step of hydrolytically reacting the solid acid material and the at least one proteinaceous material in a non-aqueous and solvent-free environment occurs at a temperature in a range of from about −5 to about 125° C.

25. The method of claim 22, wherein at least about 50% by weight of protein in the proteinaceous material is converted to a protein hydrolysate comprising at least one of an amino acid, a polypeptide, and combinations thereof.

26. The method of claim 22, wherein a fraction of the proteinaceous material remains unreacted and at least a portion of the unreacted proteinaceous material is recycled to the step of hydrolytically reacting with the solid acid material.

27. The method of claim 26, wherein at least about 90% by weight of protein in the proteinaceous material is converted to a protein hydrolysate comprising at least one of an amino acid, a polypeptide, and combinations thereof.

28-44. (canceled)

45. The method of claim 22, wherein the protein hydrolysate reaction product is produced by a mechanocatalytic reaction of the at least one proteinaceous material and the solid acid material.

46. The method of claim 22, wherein the solid acid material is selected from the group consisting of aluminosilicate materials, solid superacid materials, and combinations thereof.

47. The method of claim 46, wherein the solid acid material comprises a solid superacid material, wherein the solid superacid material comprises a solid selected from the group consisting of natural clays, natural minerals, metal oxides, metal sulfides, metal salts, and combinations thereof that has been treated with at least one of a Bronsted acid and a Lewis acid.

48. The method of claim 22, wherein the solid acid material comprises an acid treated clay material, wherein the clay material is selected from the group consisting of kaolin, bentonite, fuller's earth, and combinations thereof.

49. The method of claim 22, wherein the solid acid material has a surface acidity, as expressed by the Hammet and Dryups function value, Hₒ, of less than about 1.

50. The method of claim 22, wherein the at least one proteinaceous material and the solid acid material have a combined free water content less than about 45% by weight.

51. The method of claim 22, wherein the at least one proteinaceous material and the solid acid material are present at a weight ratio of from about 0.5:1 to about 10:1.
